(19) United States
(12) Patent Application Publication
(10) Pub. No.: US 2014/0240124 A1
(43) Pub. Date: Aug. 28, 2014

(54) METHOD AND APPARATUS FOR MONITORING, DETERMINING AND COMMUNICATING BIOMETRIC STATUSES, EMOTIONAL STATES AND MOVEMENT

(71) Applicant: Exmovere Wireless LLC, Alexandria, VA (US)

(72) Inventor: David Bychkov, Alexandria, VA (US)

(73) Assignee: Exmovere Wireless LLC, Alexandria, VA (US)

(21) Appl. No.: 14/187,287

(22) Filed: Feb. 23, 2014

Related U.S. Application Data
(60) Provisional application No. 61/768,557, filed on Feb. 25, 2013.
(60) Provisional application No. 61/768,556, filed on Feb. 25, 2013.

(51) Int. Cl. A61B 5/00

(52) U.S. Cl. CPC A61B 5/0026
USPC 340/539.12

(57) ABSTRACT

An approach is provided for collecting and processing biometric information associated with a user of a body-mounted device to determine a status of the user. The approach involves causing biometric information to be collected by way of a body-mounted device. The approach further involves causing the biometric information to be communicated to a network management system. The approach additionally involves causing one or more of the network management system and the body-mounted device to process the biometric information to determine a status of the user of the body-mounted device, the status including one or more vital signs and an emotional state of the user based on the biometric information. The approach also involves causing the status to be displayed by the body-mounted device.

### RELATED APPLICATIONS

This application is related to the following co-pending application which is hereby incorporated herein by reference in its entirety: U.S. patent application Ser. No. 12/910,840, filed Oct. 24, 2010, entitled “PERSONAL HEALTH MONITORING DEVICE,” by David Bychkov, and also claims the benefit of the earlier filing date of U.S. Provisional Patent Application No. 61/768,557, filed Feb. 25, 2013, entitled “METHOD AND APPARATUS FOR MONITORING, DETERMINING AND COMMUNICATING VITAL SIGNS, EMOTIONAL STATES AND MOVEMENT,” by David Bychkov; and U.S. Provisional Patent Application No. 61/768,556, filed Feb. 25, 2013, entitled “METHOD AND APPARATUS FOR MONITORING INFANT HEALTH AND EMOTIONAL STATE,” by David Bychkov, both of which are hereby incorporated herein in their entireties by reference under 35 U.S.C. §119(e).

### BACKGROUND

Service providers and device manufacturers (e.g., wireless, cellular, etc.) are challenged to deliver value and convenience to consumers by, for example, providing compelling network services. Such services may include determining and communicating a person's biometric status, emotional state and location.

### SOME EXAMPLE EMBODIMENTS

Therefore, there is a need for an approach to determine and communicate a biometric status, emotional state and location associated with a user of a body-mounted device.

According to one embodiment, a method comprises causing, at least in part, biometric information to be collected by way of a body-mounted device, the biometric information corresponding to a user of the body-mounted device. The method further comprises causing, at least in part, the biometric information to be communicated to a network management system. The method additionally comprises causing, at least in part, one or more of the network management system and the body-mounted device to process the biometric information to determine a status of the user of the body-mounted device. The method also comprises causing, at least in part, the status to be displayed by the body-mounted device. The status comprises one or more vital signs and an emotional state of the user based, at least in part, on the biometric information.

According to another embodiment, an apparatus comprises at least one processor, and at least one memory including computer program code for one or more computer programs, the at least one memory and the computer program code configured to, with the at least one processor, cause, at least in part, the apparatus to cause, at least in part, biometric information to be collected by way of a body-mounted device, the biometric information corresponding to a user of the body-mounted device. The apparatus is further caused to cause, at least in part, the biometric information to be communicated to a network management system. The apparatus is additionally caused to cause, at least in part, one or more of the network management system and the body-mounted device to process the biometric information to determine a status of the user of the body-mounted device. The apparatus is also caused to cause, at least in part, the status to be displayed by the body-mounted device. The status comprises one or more vital signs and an emotional state of the user based, at least in part, on the biometric information.

Exemplary embodiments are described herein. It is envisioned, however, that any system that incorporates features of any apparatus, method and/or system described herein are encompassed by the scope and spirit of the exemplary embodiments.

### BRIEF DESCRIPTION OF THE DRAWINGS

The embodiments are illustrated by way of example, and not by way of limitation, in the figures of the accompanying drawings:

FIG. 1 is a diagram of a system capable of determining and communicating a biometric status, emotional state and location associated with a user of a body-mounted device, according to one or more embodiments.

FIG. 2 is a diagram of the components of a status control platform, according to one or more embodiments.

FIG. 3 is a flowchart of a process for determining and communicating a biometric status, emotional state and location associated with a user of a body-mounted device, according to one or more embodiments.

FIG. 4 is a series of diagrams illustrating a user interface utilized in the processes of FIG. 3, according to one or more embodiments.

FIG. 5 is a diagram of a system capable of determining and communicating a biometric status, emotional state and location associated with a user of a body-mounted device, according to one or more embodiments.

FIG. 6 is a diagram of a matrix upon which generated personalized health guidance messages are based, according to one or more embodiments.

FIG. 7 is a diagram of a chip set that can be used to implement an embodiment.

### DESCRIPTION OF SOME EMBODIMENTS

Examples of a method, apparatus, and computer program for determining and communicating a biometric status, emotional state and location associated with a user of a body-mounted device are disclosed. In the following description, for the purposes of explanation, numerous specific details are set forth in order to provide a thorough understanding of the embodiments. It is apparent, however, to one skilled in the art that the embodiments may be practiced without these specific details or with an equivalent arrangement. In other instances, well-known structures and devices are shown in block diagram form in order to avoid unnecessarily obscuring the embodiments.

As used herein, the term “biometric status,” or any derivation thereof, refers to one or more of a vital sign, health status, health condition, etc.

As used herein, the term “biometric information,” or any derivation thereof, refers to one or more of a heart rate, a body temperature, a breath rate, a blood glucose level, a blood oxygen content, blood pressure, a skin hydration level, a degree of movement, an orientation of a user, or other types of suitable collected information usable to determine a biometric status or emotional state.

As used herein, the term “biometric sensor,” or any derivation thereof, refers to a device capable of collecting data associated with or determining biometric information such as an infrared (IR) sensor, a global positioning system (GPS) unit, an accelerometer, a three-axis accelerometer, a gyroscope, a thermistor sensor, an optical sensor, a pressure sensor, an audio sensor or other suitable sensor capable of collecting data associated with or determining biometric information of a user.

As used herein, the term “biosensor populated fabric” refers to any combination of a fabric configured to accommodate one or more sensors and a fabric having integrated sensory capabilities such as, but not limited to, sensors associated with any fibers of the fabric itself.

As used herein, the term “emotional state,” or any derivation thereof refers to one or more of energetic, anxious, excited, joyful, calm, relaxed, peaceful, frustrated, stressed, busy, bored, fatigued, depressed, sleeping, resting, crying, sick, happy, sad, restless, or any other type of emotional state or mood that is determinable based, at least in part, on collected biometric information.

As used herein, the term “user status” generally refers to one or more of a biometric status, an emotional state, and/or a location of a user.

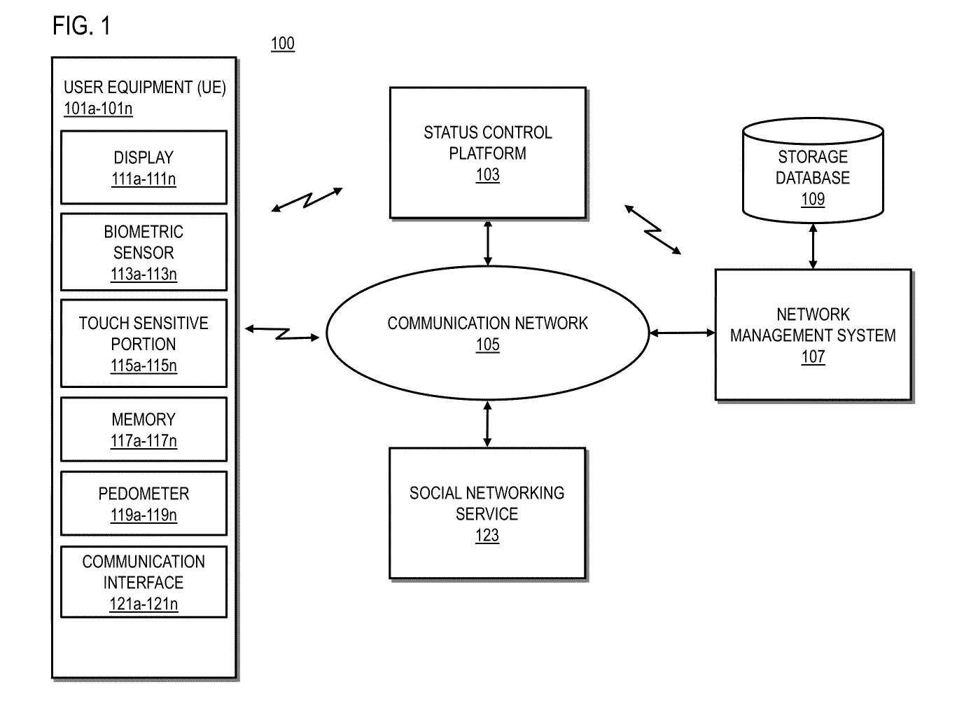

FIG. 1 is a diagram of a system capable of determining and communicating a biometric status, emotional state and location associated with a user of a body-mounted device, according to one or more embodiments. Conventional medic alert bracelets often communicate vital signs or a distress signal to a service provider. Some mobile devices are configured to provide location information to a service provider. But, service providers do not know a user's emotional state which could be used for any number of purposes such as healthcare service needs, promotional purposes, military personnel tracking, athletic performance tracking, social networking purposes, or other suitable application.

To address this problem, a system 100 of FIG. 1 introduces the capability to determine and communicate a biometric status, emotional state and location associated with a user of a body-mounted device. As shown in FIG. 1, the system 100 comprises user equipment (UE) 101a-101b (collectively referred to herein as “UE 101”) having connectivity to a status control platform 103, a network management system 107, and a social networking service 123 via a communication network 105. The status control platform 103 is any combination of a stand-alone feature independent from the UE 101 and the network management system 107, integrated with UE 101 and/or the network management system 107, or directly associated with the UE 101 and/or the network management system 107.

In some embodiments, the network management system 107 has connectivity to a storage database 109. The network management system 107, in some embodiments, is associated with a network service provider and configured to monitor and control the various functions and features available to the system 100 as a whole.

The UE 101's comprise corresponding displays 111a-111n (collectively referred to herein as “display 111”), biometric sensors 113a-113n (collectively referred to herein as “biometric sensor 113”), touch sensitive portions 115a-115n (collectively referred to as “touch sensitive portion 115”), memories 117a-117n (collectively referred to herein as “memory 117”), pedometers 119a-119n (collectively referred to herein as “pedometer 119”), and communication interfaces 121a-121n (collectively referred to herein as “communication interface 121”).

The UE 101 is a body-mounted device, or can support any type of interface to the user (such as “wearable” circuitry, etc.), that is mounted, worn, or implanted, on or in one or more of a user's wrist, arm, hand, torso, neck, head, abdomen, leg, ankle, foot, or other suitable bodily position from which biometric information is capable of being collected or sensed. Though discussed primarily as a body-mounted device, it should be noted that the UE 101 may be any type of mobile terminal, or portable terminal including a mobile handset, station, unit, device, multimedia computer, multimedia tablet, Internet node, communicator, desktop computer, laptop computer, notebook computer, netbook computer, tablet computer, personal communication system (PCS) device, personal navigation device, personal digital assistants (PDAs), audio/video player, digital camera/camcorder, positioning device, television receiver, radio broadcast receiver, electronic book device, game device, or any combination thereof, including the accessories and peripherals of these devices, or any combination thereof.

In some embodiments, if the UE 101 is wearable, the UE 101 is a body-mounted device configured to be wearable by a user of any age or gender, including infants. In embodiments, the UE 101 is configured to be a bracelet, a watch, an anklet, or other suitable bodily worn device or combination thereof. In some embodiments, the UE 101 is configured to be a biosensor populated fabric in the form of a shirt, a pair of pants, a pair of shorts, a one-piece body suit, a hat, a glove, a sock, a belt, eyewear, a necklace, a strap, or other suitable bodily worn device, or combination thereof. In some embodiments, the UE 101 comprises a tightening portion configured to facilitate consistent contact with a skin surface. The tightening portion includes, for example, an elastic material, zipper, tie, or other suitable fastener that facilitates conforming one or more portions of the UE 101 to a user's body. The tightening portion is positioned on the UE 101 in a location that corresponds to a desired data reception area such as, but not limited to, a waist line, stomach, chest, back, temple, wrist, finger tip, palm, ankle, neck, thigh, calf, arm, forehead, etc. In some embodiments, if the UE 101 comprises a biosensor populated fabric, the biosensor populated fabric includes a communication port configured to facilitate external connectivity to the biometric sensor 113, the external connectivity being one or more of physical connectivity or wireless connectivity.

According to various embodiments, the biometric sensors 113 comprise one or more devices capable of collecting data associated with or determining biometric information such as an infrared (IR) sensor, a global positioning system (GPS) unit, an accelerometer, a three-axis accelerometer, a gyroscope, a thermistor sensor, an optical sensor, a pressure sensor, an audio sensor or other suitable sensor capable of collecting data associated with or determining biometric information of a user. In some embodiments, at least one biometric sensor 113 is configured to contact a skin surface of a user of the UE 101. In some embodiments, at least one biometric sensor 113 is, for example, a dry sensor configured to collect biometric information associated with the user by way of the contact with the skin surface without the need for conductive liquids or gels.

According to various embodiments, collected biometric information corresponds to a user of the UE 101. The biometric information includes, for example, any biometric information that could be used to determine a biometric status or emotional state of the user of the UE 101. In some embodiments, the biometric information includes one or more of a heart rate, a body temperature, a breath rate, a blood oxygen content, blood pressure, a skin hydration level, a degree of movement, an orientation of the user, or other types of suitable collected information from which a user biometric status or emotional state is determined.

According to various embodiments, for example, the status control platform 103 determines one or more of heart rate, body temperature, skin temperature, blood oxygen content, blood pressure, and blood glucose level by processing data collected by way of a biometric sensor 113 that is an IR sensor. In some embodiments, the status control platform 103 determines one or more of location, body position, orientation, movement, degree of movement, or other location or movement-based information by processing data collected by way of a biometric sensor 113 that is any of a GPS unit, an accelerometer, a three-axis accelerometer, or a gyroscope.

A determinable emotional state of the user includes one or more of energetic, anxious, excited, joyful, calm, relaxed, peaceful, frustrated, stressed, busy, bored, fatigued, depressed, sleeping, resting, crying, sick, happy, sad, restless, or any other type of emotional state or mood that is determinable based, at least in part, on the collected biometric information. In other words, the emotional state is any of a behavior or a state of mind. In some embodiments, the biometric information is used to determine a degree, level and/or duration of pain experienced by a user of the UE 101. In some embodiments, the biometric information is used to determine a level, degree and/or duration of depression and/or of a mood swing of a user of the UE 101.

In one or more embodiments, the status control platform 103 causes, at least in part, the UE 101 to collect the above-discussed biometric information by way of one or more biometric sensors 113 and transmit the biometric information to the status control platform 103 and/or the network management system 107 for processing. Alternatively, the status control platform 103 causes the biometric information to be directly communicated to the social networking service 123. The status control platform 103 processes the received biometric information to determine a biometric status and/or an emotional state of the user based, at least in part, on the biometric information.

In some embodiments, the status control platform 103 causes, at least in part, a determined user status comprising one or more of a determined biometric status and an emotional state to be displayed on at least the UE 101 using the display 111. The user status is indicated by any message, graphic, multimedia message, alert sound, haptic response such as a vibration, or other suitable indication that corresponds to one or more preferences that are optionally set regarding a user interface accessible by way of display 111 that indicates a user status of the user of the UE 101.

In some embodiments, the UE 101 is used to facilitate tracking various biometric information or vital signs, calorie consumption, calorie burn information, craving information based on particular food consumption data, calorie consumption data, and any determined emotional or behavioral state. Such information is tracked and stored by the UE 101 and/or the network management system 107 for later review or processing that determines, for example, a degree, level and/or duration of a craving for a particular food, need, or desire.

The UE 101 is configured to communicate with the network management system 107 by way of the communication interface 121. The UE 101 is configured to include any number of communication interfaces 121 that comprise one or more of a transmitter, a receiver, a subscriber identification module, a near field communication interface, a USB interface, a global positioning unit, a wireless network communication unit, or other suitable communication interface capable of transferring or transmitting and/or receiving data.

The UE 101 determines location information associated with the UE 101, for example, by way of a communication interface 121 that is a GPS unit, or by way of location information provided by way of the communication network 105 such as one or more services made available by a communication network 105 provider or by the network management system 107. The UE 101 shares determined location information by transmitting the location information to the network management system 107, or the UE 101 shares data to be processed by the network management system 107 to determine location information. In some embodiments, the location information is shared with the social networking service 123. In some embodiments, the location information is used to determine the emotional state and/or the user status of the user of the UE 101.

In some embodiments, as discussed above, the UE 101 includes a pedometer 119. The pedometer 119 is used to collect data pertaining to the above-discussed location information. The pedometer 119 is also capable of being used to determine a number of steps taken by a user of the UE 101. The UE 101, if outfitted with the pedometer 119, provides data collected by the pedometer 119 to the network management system 107 which determines calories burned, for example, based, at least in part, on the data collected by the pedometer 119. In some embodiments, the UE 101 and/or the network management system 107 are configured to process data collected by the pedometer 119 for location determination and/or calorie usage. Calorie usage, for example, is helpful in directing a user of the UE 101 by way of displayed messages via the display 111, how to meet specified dietary goals or consumption schedules without having to carry a large personal computer or smart phone.

In some embodiments, the status control platform 103 causes, at least in part, data such as the collected biometric information, determined location information, preferences, cravings, consumption data, pain related data, and/or the biometric status determinations or emotional state to be stored in the storage database 109 and/or the memory 117. The stored data is processed to determine a trend in the user's behavior and to generate a report log or a message that indicates any concerns or alerts that are of interest based on any determined trends.

In some embodiments, the network management system 107 has connectivity to an emergency care or health service provider, and based on preset rules or settings, is caused by the status control platform 103 to contact the emergency care or health service provider if a user's vital signs or emotional state such as sickness or depression breach a particular alert threshold level.

In some embodiments, the UE 101 provides messages that are textual or multimedia-based that alert a user to particular shopping incentives or events that are relevant to a determined current location of the UE 101, or that are relevant to a habitual, past, or projected future location of the user of the UE 101 based, at least in part, on the collected location information and/or emotional state determined and stored in the storage database 109. For example, the network management system 107 is configured to estimate, based on a user's current mental state and projected location at a time in the future, or current location, a user's desire, or craving, and causes an advertisement or promotional notification for a nearby ice cream shop, sporting goods store, general apparel store, or restaurant to appear on the display 111.

As discussed above, in some embodiments, the UE 101 includes a touch sensitive portion 115. The touch sensitive portion 115 comprises one or more of a button that is raised, ribbed or a dimple on a surface of the UE 101, or a touch sensor that is flush with a surface of the UE 101. In some embodiments, the touch sensitive portion 115 is configured to cause the UE 101 transmit a distress signal based, at least in part, on an input received by way of the touch sensitive portion 115. In some embodiments, the touch sensitive portion 115 comprises at least a portion of display 111 such that the display 111, or an entirety of the display 111, such that display 111 is a touch screen display by which a user interacts with a user interface provided via display 111 or operating system.

In one or more embodiments, the network management system 107 has an interface capable of accessing the UE 101 to facilitate configuring the UE 101 remotely by way of the network management system 107.

In some embodiments, the system 100 is configured to provide personalized health guidance. For example, the status control platform 103 is configured to encourage a user of UE 101, based on collected biometric information, location information, or determined emotional state, to be joyful, work toward having a normal body mass index (BMI), be less stressed, be more active, eat better, take vitamins, to consume a proper amount of water, or to direct the user to adopt another behavioral change that affects the collected biometric information, biometric status and/or emotional state of the user or to achieve another health or emotionally directed goal.

For example, in some embodiments, the system 100 teaches a user to control skin temperature, heart rate and sweating through messages generated by the status control platform 103 that direct a user how to change the user's behavior or current biometric state based on collected biometric information, to reduce, for example, chronic pain, anxiety and/or depression.

In some embodiments, to encourage personalized health guidance, the status control platform 103 generates messages to be displayed by the UE 101 via display 111 to teach or direct a user to maintain circulation to the hands and feet, to reduce stress-activated sweating and shaking, to understand emotional changes related to heart rate, to drink enough water and to eat correctly by suggesting what to eat and when, suggesting which vitamins to take, to get fresh air and sunlight, and/or to keep the body in motion with exercise stretching and walking.

For example, based on a user's input height and weight, as well as the biometric information and/or location information collected by way of UE 101, the status control platform 103 is configured to determine has a low BMI, a high BMI, or a normal BMI. In some embodiments, based on one or more user inputs, the biometric information, and/or the location information collected by the UE 101, the status control platform 103 is configured to determine if the user is sedentary (e.g., the user takes less than 5,000 steps per day) based on a determined number of steps collected by the pedometer 119, active (e.g., the user takes more than 5,000 less than 10,000 steps per day), a power walker (e.g., the user more than 10,000 steps per day), energetic, excited, joyful, calm, relaxed, peaceful, busy, stressed, frustrated, bored, fatigued, or distressed.

In some embodiments, the status control platform 103 determines user emotional states or biometric statuses based on a comparison of the collected biometric information and/or location information to default values that correlate particular combinations of biometric information, vital signs, locations, movement amounts, types of movements, changes of biometric information, changes in vital signs, changes in location, changes in movement amounts, changes between types of movements, time durations between or of types of movements, and/or other suitable and determinable trends based on collected data and/or times with respect to various emotional states and biometric statuses.

In some embodiments, the status control platform 103 is configured to process user input information received via UE 101 that describes an emotional state or level of pain the user is experiencing. The status control platform 103 records the biometric information and location information that corresponds to the user input information that describes the user biometric status, and stores that information in storage database 109. Then, the UE 101 collects more biometric information and location information, the network management system develops a user profile. Changes in the collected biometric information and location information are compared to the developed user profile, and the emotional state or biometric status of the user is determined based, at least in part, on the comparison of the collected biometric information and location information to the user profile.

Based, for example, on the default values and/or the user profile, the status control platform 103 is capable of distinguishing certain bodily needs from those that raise concern. For example, the status control platform 103 is configured to determine, based on the biometric information, if a user is drinking enough water, is dehydrated, or is over-hydrated. The status control platform 103 is also configured to determine if a user is stressed, for example, based on a detected skin moisture or sweat level. To distinguish between a skin moisture level that might be considered to correspond to both over-hydration and stress (e.g., because the user is sweating), the status control platform 103 also considers other biometric information such as heart rate, blood pressure, and determined movement to discern based on the combination of skin moisture level with other biometric information whether the user is over-hydrated or is in fact stressed.

In some embodiments, if the status control platform determines a user is stressed or is experiencing anxiety, the status control platform 103 generates messages to be displayed by the UE 101 via display 111 messages that direct a user to calm down, slow their breathing rate, move differently, eat differently, or other suitable suggestion to reduce the determined stress or anxiety level. If the determined stress or anxiety level is not reduced within a predetermined period of time, then the status control platform 103, in some embodiments, generates a message suggesting that the user seek medical attention.

In some embodiments, if a user inputs one or more specified goals such as losing weight, being more active, eating better, being more productive, or living a less stressful life, the status control platform 103 determines if the user is making progress toward the specified goals based on the developed user profile. For example, the status control platform 103 is configured to analyze the determined trends and/or stored collected biometric, emotional state and/or location information, and determines what the user should do to make more, or better, progress to cause the trending information to move toward a predetermined trend line associated with the specified goal. The status control platform 103 is also configured to generate an instruction or suggestion message to be displayed by the UE 101 via display 111 for the user to follow to help the user achieve the specified goal.

In some embodiments, the system 100 is configured to provide suggestive messages to provide biofeedback to help a user fight depression. For example, the status control platform 103, in some embodiments, is configured to determine a user's emotional state is depressed either based on collected biometric information and/or location information, and to generate suggestion messages to be displayed by UE 101 via display 111 to improve the user's mood, along with encouraging ideas that increase overall health.

In some embodiments, to combat depression, the status control platform 103 causes a message to be displayed via UE 101 that instructs a user to forcefully laugh to change the user's heart rate and determines the effect based on the collected biometric information. In some embodiments, the status control platform 103 generates a message directing the user to go outside, or to at least sit by a window to get Vitamin D from the sun. The status control platform 103, for example, uses the location information to determine if the user is indoors or outdoors and to determine if the user followed the instruction. In some embodiments, the status control platform 103 generates a message suggesting the user walk at least a few thousand steps, and uses the location information and the pedometer 119 to determine if the user followed the instruction. In some embodiments, the status control platform 103 generates a message suggesting foods that improve mood, like salmon, chocolate, or food the user historically has eaten to improve the determined emotional state.

In some embodiments, the system 100 is configured to help a user to rehabilitate muscles, joints and nerves. For example, the system 100 helps users that spend time walking, climbing steps, carrying objects, standing in subways, waiting in line and running after taxis to manage fatigue and increase physical performance. In some embodiments, the status control platform 103 determines if a user is drinking enough water and generates a message suggesting the user drink water if the status control platform 103 determines, based on the collected biometric information, that the user has a skin moisture percentage that is less than about 50%.

In some embodiments, the status control platform 103 determines, based on a determined activity level, whether the user has stretched the user's muscles thoroughly or for a long enough period following a workout. In some embodiments, the status control platform 103 is configured to generate a message instructing the user to stretch, based on a determined activity level and a determined location. In some embodiments, if the status control platform 103 determines the user needs to stretch, but based on the determined activity level based on the collected biometric information and location information, determines the user is not in a location that makes stretching an acceptable activity, the status control platform 103 generates a message instructing the user to stretch by checking on footwear and to adjust socks. Alternatively, if the status control platform 103 determines the user has poor blood circulation in the legs or feet, the status control platform 103 similarly generates a message instructing the user to check on footwear and to adjust socks.

In some embodiments, the status control platform 103 determines how much time the user has taken between eating and increasing the user's activity level, ensuring the user has had enough time to digest food before beginning a particular activity. If the user has not, the status control platform 103 generates a message suggesting the user wait a specified period of time (such as one-hour before swimming) after eating.

In some embodiments, the system 100 is configured to help a user of the UE 101 combat migraine headaches. For example, migraines can be treated by promoting circulation to the hands, and through thermal biofeedback. Based on collected biometric information, such as body temperature, and a determination that the user is experiencing pain or a migraine headache, the status control platform 103 generates a message instructing the user to start deep breathing and to relax, promoting the wrists and hands to become warmer. The status control platform 103 determines if the user has followed the instruction based on the collected biometric information.

In some embodiments, the system 100 is configured to help a user slow compulsions, cravings, understand their origin, and make healthier choices. In some embodiments, the status control platform 103 helps users battle addictions, eating disorders and general cravings by generating messages suggesting alternative behavior. For example, if a user suffers from drug addiction and the status control platform 103 determines the user is in a frustrated emotional state, the status control platform 103 generates a message that suggests one or more ways to calm down and regain self-control. For example, the status control platform 103, in some embodiments, generates a message suggesting the user move the user's arms in circles until the status control platform 103 determines, based on the biometric information, that the user successfully raises the determined body temperature at least 2 degrees Celsius. In some embodiments, the status control platform 103 generates a message suggesting the user breathe deeply until the status control platform 103 determines, based on the biometric information, that the user's skin moisture is reduced by 10% or more from the current determined skin moisture level. In some embodiments, the touch sensitive portion 115 is configured to directly contact a counselor or specialist on call to help the user in a time of need.

In some embodiments, the system 100 is configured to help a user to modulate the user's fight or flight response, during stressful situations. For example, the status control platform 103 is configured to help a user to take control of the user's emotions during panic attacks and to help the user reduce chronic stress. For example, the status control platform 103 generates a message to help a user in the case of airplane turbulence suggesting the user visualize a safe, peaceful place, to pour a cup of water and observe how, even if the water moves during turbulence, it stays in the cup, and/or to suggest the user watch the user's heart rate drop by 5-10 beats per minute.

In some embodiments, the status control platform 103 provides users with metrics or trends for stress reduction, pain management, self-control and physical fitness. As discussed, the status control platform 103 and/or the network management service 107 develop a user profile that tracks historical data so that the status control platform 103 learns from the user's behavior and trending biometric information so that the status control platform 103 generates personalized messages associated with determined biometric information, biometric status and/or emotional state.

In some embodiments, the system 100 is configured to help a user stay focused and increase production. For example, if the status control platform 103 determines a user is stressed based on the biometric information, the status control platform 103 generates a message suggesting the user visualize and mentally rehearse stressful situations in great detail and notice the effect on palm and finger moisture. In some embodiments, the status control platform 103 generates a message suggesting the user write down which specific thoughts or ideas increase the stress response in the skin. In some embodiments, the status control platform 103 generates a message suggesting the user try to experience very stressful sounds or images and while keeping skin moisture from increasing or decreasing. The lower the user's palm moisture, for example, and slower it changes, the more ready the user is to handle stressful situations.

In some embodiments, the system 100 is configured to help a user achieve an effective workout. For example, if used during Yoga, Tai Chi and martial arts training, the status control platform 103, based on a determination that the user is wearing the multiple UE 101's (e.g., a UE 101a on one ankle and a UE 101b on the other ankle), and is working out, generates a message suggesting the user practice warming the user's feet with meditation only. During Bikram yoga, the status control platform 103 is configured to generate a message suggesting a user practice maintaining, and even decreasing the user's skin moisture levels despite the intense heat.

In some embodiments, the status control platform 103 is configured to determine the user is working out, and to determine if the user is sweating during the workout based, for example, on the biometric information, and generates a message reminding the user to drink water during and after the workout. In some embodiments, the status control platform 103 determines the user is working out, and based on biometric information such as body temperature information, the status control platform 103 determines if the user has warmed up the user's muscles to a predetermined level or for a predetermined time. Based on a determination that the user is working out and a determination that the user needs to warm up the user's muscles, the status control platform 103 generates a message suggesting the user warm up and/or cool down properly before and after the workout.

In some embodiments, the system 100 is configured to be an infant health and emotional state monitor. Conventional baby monitors often provide only audio and/or visual data regarding an infant. Parents often use conventional baby monitors to determine whether a baby is crying, sleeping, behaving and generally at rest, etc. Parents are often concerned about the biometric status and emotional state of their baby when they are in another room, away from home, when the baby is in the care of another individual and the like. Conventional baby monitors do not provide this kind of information.

In some embodiments, the status control platform 103 causes, at least in part, a first UE 101a to collect the above-discussed biometric information and transmit the biometric information to a second UE 101b that acts as a base station. The base station UE 101b, in some embodiments, is configured to process the biometric information, or the base station UE 101b communicates the received biometric information to a third UE 101c that acts as a receiver or the network management system 107 for processing. The receiver UE 101c displays user status messages regarding the user of the UE 101a.

Alternatively, the status control platform 103 causes the biometric information to be directly communicated to the receiver UE 101c and/or the network management system 107 if, for example, the UE 101a comprises a transmitter capable of communicating directly with the receiver UE 101c and/or the network management system 107. The system 100 makes it possible, for example, for a parent to remotely check on the status of their baby. Based on a determined emotional state associated with the collected biometric information, the system 100 makes it possible for a parent to determine from afar if the baby is sick, sleeping, restless, crying, happy, sad, excited, or is experiencing another emotional state, or is located in an unexpected location. Similarly, a parent may want to have their baby's vitals presented to them by the receiver UE 101a, of if the UE 101a worn by the baby is configured having display 111, directly by the UE 101a.

In some embodiments, a base station UE 101b is optionally configured to charge a battery of the UE 101a and/or the UE 101c. The base station UE 101b, if the system 100 is so equipped, is configured to facilitate communication between the base station UE 101b, the receiver UE 101c, the network management system 107, and/or the status control platform 103, using the identical or different communication channels, technologies or frequencies. But, in some embodiments, while the base station UE 101b is configured to communicate with the network management system 107 by way of any cellular service network or any of the communication medium discussed with regard to communication network 105, the base station UE 101b and the UE 101a are separately configured to communicate with one another via FM frequencies, the base station UE 101b and the UE 101a are configured to communicate via WiFi or Near Field Communication with one another, or by way of another suitable short-range communication medium.

By way of example, the communication network 105 of system 100 includes a direct wired connection, or one or more networks such as a wired data network, a wireless network, a telephony network, or any combination thereof. It is contemplated that the data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (WiFi), WiGig, wireless LAN (WLAN), Bluetooth®, Internet Protocol (IP) data casting, satellite, mobile ad-hoc network (MANET), and the like, or any combination thereof.

By way of example, the UE 101, status control platform 103, network management system 107, and social networking service 123 communicate with each other and other components of the communication network 105 using well known, new or still developing protocols. In this context, a protocol includes a set of rules defining how the network nodes within the communication network 105 interact with each other based on information sent over the communication links. The protocols are effective at different layers of operation within each node, from generating and receiving physical signals of various types, to selecting a link for transferring those signals, to the format of information indicated by those signals, to identifying which software application executing on a computer system sends or receives the information. The conceptually different layers of protocols for exchanging information over a network are described in the Open Systems Interconnection (OSI) Reference Model.

Communications between the network nodes are typically effected by exchanging discrete packets of data. Each packet typically comprises (1) header information associated with a particular protocol, and (2) payload information that follows the header information and contains information that may be processed independently of that particular protocol. In some protocols, the packet includes (3) trailer information following the payload and indicating the end of the payload information. The header includes information such as the source of the packet, its destination, the length of the payload, and other properties used by the protocol. Often, the data in the payload for the particular protocol includes a header and payload for a different protocol associated with a different, higher layer of the OSI Reference Model. The header for a particular protocol typically indicates a type for the next protocol contained in its payload. The higher layer protocol is said to be encapsulated in the lower layer protocol. The headers included in a packet traversing multiple heterogeneous networks, such as the Internet, typically include a physical (layer 1) header, a data-link (layer 2) header, an internetwork (layer 3) header and a transport (layer 4) header, and various application (layer 5, layer 6 and layer 7) headers as defined by the OSI Reference Model.

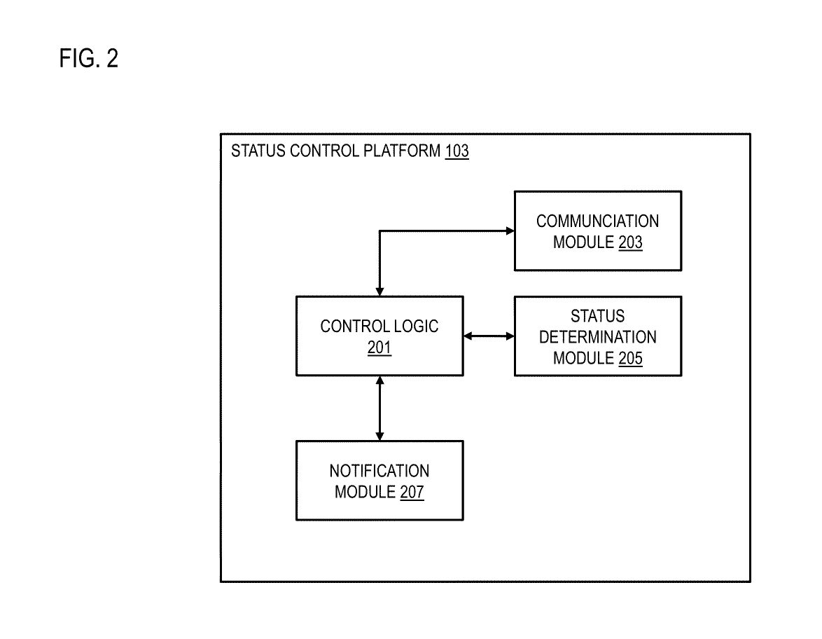

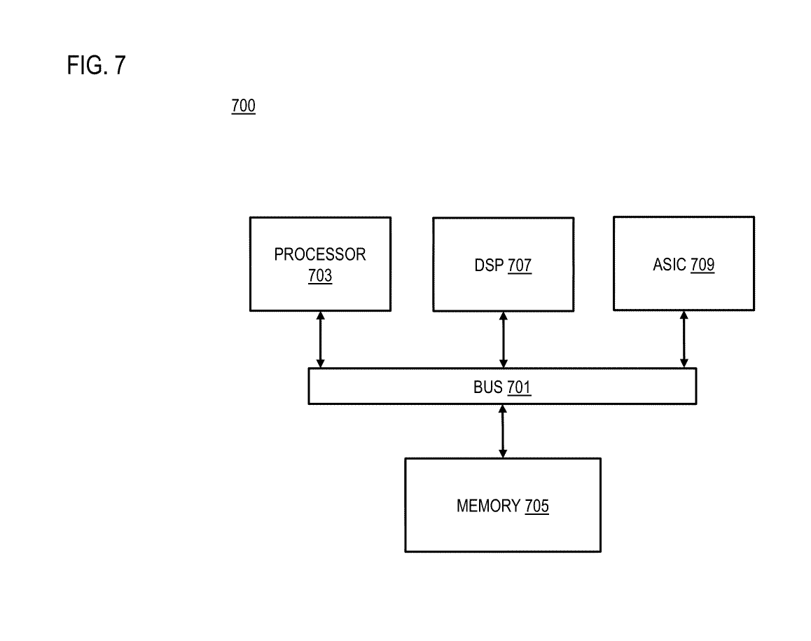

FIG. 2 is a diagram of the components of a status control platform 103, according to one or more embodiments. In some embodiments, the status control platform 103 includes a set of instructions that are executed by a processor such as processor 703 discussed with respect to FIG. 7. In other embodiments, the status control platform 103 is embodied as a special purpose processor configured specifically to implement the status control platform 103. By way of example, the status control platform 103 includes one or more components for determining and communicating a biometric status, emotional state and location associated with a user of a body-mounted device. It is contemplated that the functions of these components may be combined in one or more components or performed by other components of equivalent functionality. The status control platform includes a control logic 201, a communication module 203, a status determination module 205, and a notification module 207.

In some embodiments, the control logic 201 causes, by way of the communication module 203, the UE 101 (FIG. 1) to collect biometric information and location information and communicate that biometric information and location information to one or more of the status control platform 103, the network management system 107 (FIG. 1) and the social networking service 123. The control logic 201 also causes, at least in part, the status determination module 205 to one or more of determine a biometric status of the user of the UE 101 based on received biometric information, an emotional state of the user based on received biometric information and/or location information, or cause the UE 101 and/or the network management system 107 to process any received biometric information and/or location information to determine a biometric status and/or emotional state of the user of the UE 101. The user status, as discussed above, comprises one or more of a vital sign or biometric status, biometric information, location information, craving, level of pain, desire, or emotional state of the user based on the aforementioned collected and/or determined information.

The status determination module 205 also causes the UE 101 and/or the network management system 107 to communicate the determined status to the status control platform 103. Based on the communicated determined status, the control logic 201 causes, at least in part, the notification module 207 to cause the network management system 107 to generate a notification message, alert, or other suitable message relating to the determined status. In other embodiments, the notification module 207 itself generates an alert or message relating to the determined status. The notification module 207, accordingly, causes the generated alert or message, regardless of source, to be communicated to the UE 101 and/or the network management system 107. In some embodiments, the notification module 207 causes a user interface associated with displaying the determined status to be updated. In some embodiments, the notification module 207, for example, also causes emergency messages to be sent to an emergency care or health service provider.

In some embodiments, the control logic 201 causes, by way of the communication module 203, the determined status or biometric information to be stored by the network management system 107 in the storage database 109 (FIG. 1) or the memory 117 (FIG. 1) for later recall or log report production.

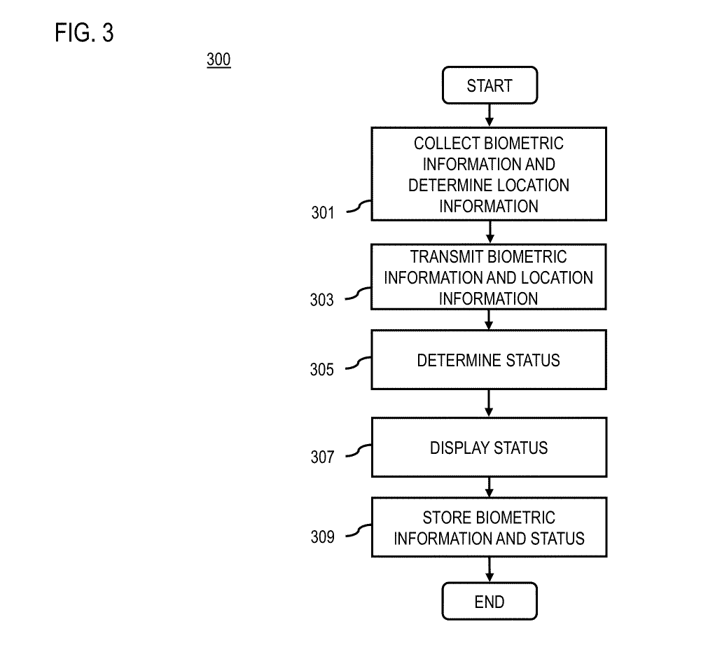

FIG. 3 is a flowchart of a process for determining and communicating a biometric status, emotional state and location associated with a user of a body-mounted device, according to one or more embodiments. The status control platform 103 performs the process 300 and is implemented in or by, for instance, a chip set including a processor and a memory as shown in FIG. 7. In step 301, the status control platform 103 causes, at least in part, biometric information to be collected by way of a body-mounted device, the biometric information corresponding to a user of the body-mounted device. The status control platform also causes location information associated with the body-mounted device to be determined. Then, in step 303, the status control platform 103 causes, at least in part, the biometric information and the location information to be communicated to a network management system.

Next, in step 305, the status control platform 103 causes, at least in part, the biometric information and the location information to be processed by one or more of the body-mounted device and the network management system to determine a status of the user of the body-mounted device. The status of the user comprises one or more of a vital sign and emotional state of the user of the body-mounted device. The emotional state is based, at least in part on one or more of the biometric information and the location information. The process continues to step 307 in which the status control platform 103 causes, at least in part, the status to be displayed by at least the body-mounted device.

Then, in step 309, the status control platform 103 causes, at least in part, the biometric information, the location information, and the status to be stored in at least one memory associated with the body-mounted device and/or the network management system.

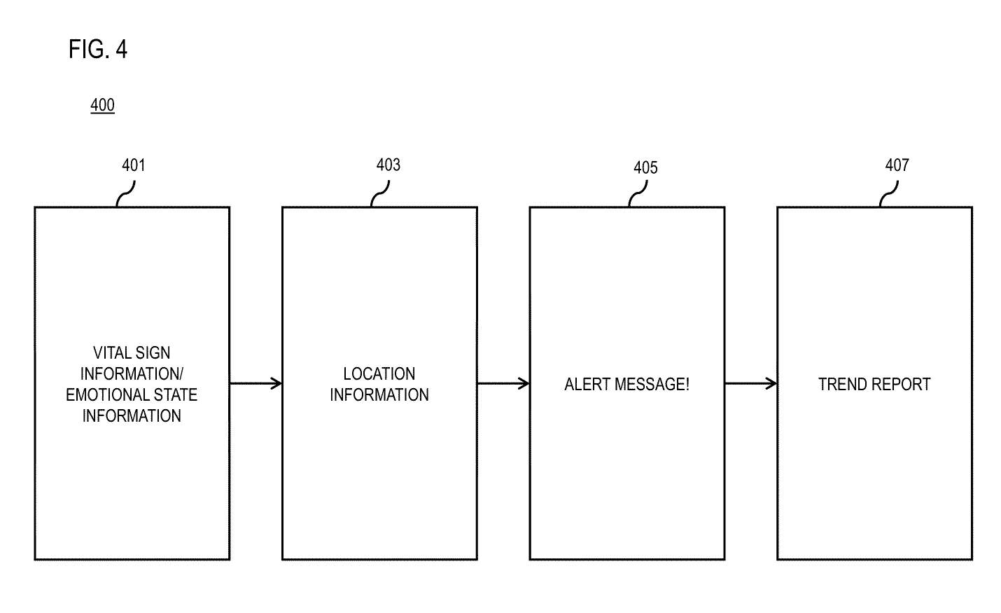

FIG. 4 illustrates an example user interface 400 utilized in the processes of FIG. 3, according to one or more embodiments. Some embodiments include any number of user interface displays that are arranged or combined in any order. Example user interface displays is viewable, for example, by way of the UE 101 (FIG. 1) via display 111 (FIG. 1), a terminal associated with the network management system 107 (FIG. 1), or a mobile device other than the UE 101 such as a second UE 101 configured to act as a receiver having an application configured to communicate with the system 100 (FIG. 1). In some embodiments, a user interface display 401 is used to provide determined vital sign information and/or emotional state information such as collected biometric information, determined emotional state, or status. User interface display 403 indicates location information of the UE 101. User interface display 405 illustrates an alert message received based on a determination that an alert threshold is triggered, the alert threshold being associated with a status, vital sign, emotional state, location information, trend information-based prediction, or promotional message. User interface display 407 illustrates an example trend report that is based, for example, on any determined trends in biometric status, vital sign, location or emotional state status that is based on stored biometric information, location information, calorie consumption data, calorie usage data, determined user status information, pain logging, or other suitable collected data.

In some embodiments, the user interface 400 also displays messages generated by the status control platform 103 by way of the various user interfaces accessible using user interface 400 that optionally include messages related to event logging, pill minder capabilities, contacts management, language options, the ability to change one or more backgrounds or wallpapers as viewed by way of the user interface such as a clock format or color scheme, for example. In some embodiments, the various user interfaces accessible by way of user interface 400 facilitate communications between one or more UE 101's directly, for example.

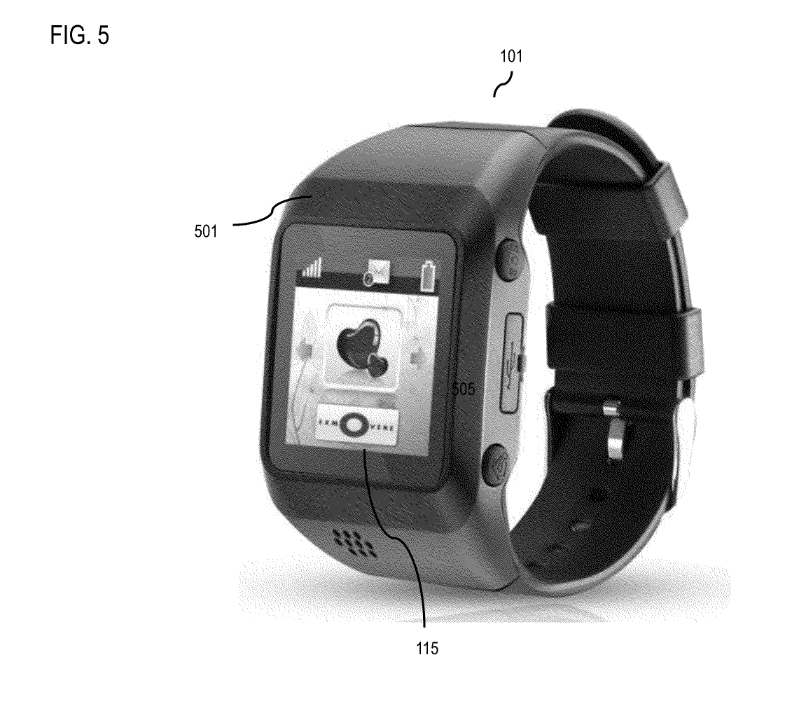

FIG. 5 illustrates an example embodiment of the UE 101. In this example, the UE 101 is a body-mounted device 501 having network connectivity to the communication network 105, discussed above. The body-mounted device 501 is configured to collect biometric information, determine location information, and one or more of provide data to the above-discussed network management system 107 to determine and track the biometric information, location information and an emotional state.

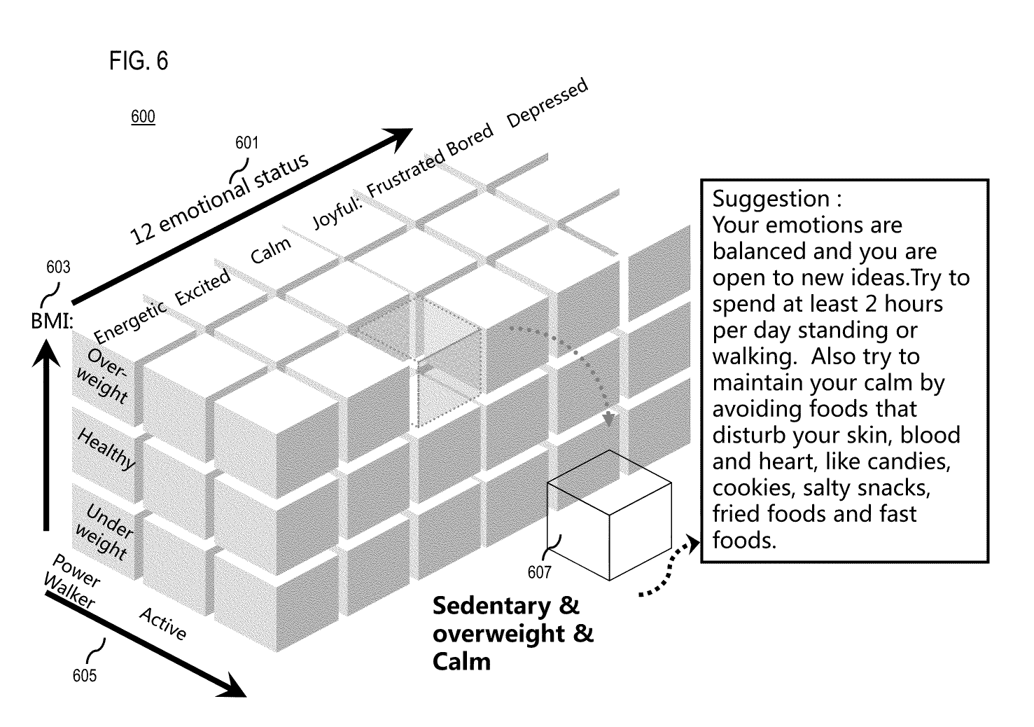

FIG. 6 is a diagram of a three-dimensional matrix 600 upon which a personalized health guidance message is based, according to one or more embodiments.

The matrix 600 includes a series of emotional states 601, a series of BMI's 603, and a series of activity levels 605 in the x, y, and z axes of the matrix 600. The status control platform 103 determines a message to be displayed by UE 101 (FIG. 1) via display 111 based on a combination of determined emotional state, BMI, and activity level.

For example, if a user is determined to be sedentary, overweight, and calm based on the collected biometric information and location information, the status control platform 103 suggests a preset message 607 that corresponds to the identified user status. In this example embodiment, matrix 600 includes twelve emotional states 601, three BMI's 603, and three activity levels 605. As such, the matrix 600 makes it possible for the status control platform to suggest 108 different preset messages based on a determined combination of determined emotional state 601, BMI 603 and activity level 605.

In some embodiments, the matrix 600 includes a greater or lesser number of emotional states 601, BMI's 603 and/or activity levels 605. Similarly, the matrix 600, in some embodiments, is alternatively configured to include different types of activities, locations, types of biometric information, emotional states, or other suitable combinations of determined behavioral states and/or health statuses that are suitable for being combined in matrix form to enable the status control platform 103 to cause a corresponding message to be displayed to a user by UE 101 via display 111.

In some embodiments, the status control platform 103 uses combinations of more than one matrix 600 interlaced with one or more other matrixes to provide a message for the user. For example, if one matrix generates a first result, that first result is input into a second matrix, and based on the first result and one or more other determined attributes such as diet and location that are input into the second matrix, the status control platform 103 determines a second result that is used to generate the message to be displayed by UE 101 via display 111.

The processes described herein for determining and communicating a biometric status, emotional state and location associated with a user of a body-mounted device may be advantageously implemented via software, hardware, firmware or a combination of software and/or firmware and/or hardware. For example, the processes described herein, may be advantageously implemented via processor(s), Digital Signal Processing (DSP) chip, an Application Specific Integrated Circuit (ASIC), Field Programmable Gate Arrays (FPGAs), etc. Such exemplary hardware for performing the described functions is detailed below.

FIG. 7 illustrates a chip set or chip 700 upon which an embodiment may be implemented. Chip set 700 is programmed to determine and communicate a biometric status, emotional state and location associated with a user of a body-mounted device as described herein may include, for example, bus 701, processor 703, memory 705, DSP 707 and ASIC 709 components.

The processor 703 and memory 705 may be incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set 700 can be implemented in a single chip. It is further contemplated that in certain embodiments the chip set or chip 700 can be implemented as a single “system on a chip.” It is further contemplated that in certain embodiments a separate ASIC would not be used, for example, and that all relevant functions as disclosed herein would be performed by a processor or processors. Chip set or chip 700, or a portion thereof, constitutes a means for performing one or more steps of determining and communicating a biometric status, emotional state and location associated with a user of a body-mounted device.

In one or more embodiments, the chip set or chip 700 includes a communication mechanism such as bus 701 for passing information among the components of the chip set 700. Processor 703 has connectivity to the bus 701 to execute instructions and process information stored in, for example, a memory 705. The processor 703 may include one or more processing cores with each core configured to perform independently. A multi-core processor enables multiprocessing within a single physical package. Examples of a multi-core processor include two, four, eight, or greater numbers of processing cores. Alternatively or in addition, the processor 703 may include one or more microprocessors configured in tandem via the bus 701 to enable independent execution of instructions, pipelining, and multithreading. The processor 703 may also be accompanied with one or more specialized components to perform certain processing functions and tasks such as one or more digital signal processors (DSP) 707, or one or more application-specific integrated circuits (ASIC) 709. A DSP 707 typically is configured to process real-world signals (e.g., sound) in real time independently of the processor 703. Similarly, an ASIC 709 can be configured to performed specialized functions not easily performed by a more general purpose processor. Other specialized components to aid in performing the functions described herein may include one or more field programmable gate arrays (FPGA), one or more controllers, or one or more other special-purpose computer chips.

In one or more embodiments, the processor (or multiple processors) 703 performs a set of operations on information as specified by computer program code related to determining and communicating a biometric status, emotional state and location associated with a user of a body-mounted device. The computer program code is a set of instructions or statements providing instructions for the operation of the processor and/or the computer system to perform specified functions. The code, for example, may be written in a computer programming language that is compiled into a native instruction set of the processor. The code may also be written directly using the native instruction set (e.g., machine language). The set of operations include bringing information in from the bus 701 and placing information on the bus 701. The set of operations also typically include comparing two or more units of information, shifting positions of units of information, and combining two or more units of information, such as by addition or multiplication or logical operations like OR, exclusive OR (XOR), and AND. Each operation of the set of operations that can be performed by the processor is represented to the processor by information called instructions, such as an operation code of one or more digits. A sequence of operations to be executed by the processor 703, such as a sequence of operation codes, constitute processor instructions, also called computer system instructions or, simply, computer instructions. Processors may be implemented as mechanical, electrical, magnetic, optical, chemical or quantum components, among others, alone or in combination.

The processor 703 and accompanying components have connectivity to the memory 705 via the bus 701. The memory 705 may include one or more of dynamic memory (e.g., RAM, magnetic disk, writable optical disk, etc.) and static memory (e.g., ROM, CD-ROM, etc.) for storing executable instructions that when executed perform the steps described herein to determine and communicate a biometric status, emotional state and location associated with a user of a body-mounted device. The memory 705 also stores the data associated with or generated by the execution of the steps.

In one or more embodiments, the memory 705, such as a random access memory (RAM) or any other dynamic storage device, stores information including processor instructions for determining and communicating a biometric status, emotional state and location associated with a user of a body-mounted device. Dynamic memory allows information stored therein to be changed by system 100. RAM allows a unit of information stored at a location called a memory address to be stored and retrieved independently of information at neighboring addresses. The memory 705 is also used by the processor 703 to store temporary values during execution of processor instructions. The memory 705 may also be a read only memory (ROM) or any other static storage device coupled to the bus 701 for storing static information, including instructions, that is not changed by the system 100. Some memory is composed of volatile storage that loses the information stored thereon when power is lost. The memory 705 may also be a non-volatile (persistent) storage device, such as a magnetic disk, optical disk or flash card, for storing information, including instructions, that persists even when the system 100 is turned off or otherwise loses power.

The term “computer-readable medium” as used herein refers to any medium that participates in providing information to processor 703, including instructions for execution. Such a medium may take many forms, including, but not limited to computer-readable storage medium (e.g., non-volatile media, volatile media), and transmission media. Non-volatile media includes, for example, optical or magnetic disks. Volatile media include, for example, dynamic memory. Transmission media include, for example, twisted pair cables, coaxial cables, copper wire, fiber optic cables, and carrier waves that travel through space without wires or cables, such as acoustic waves and electromagnetic waves, including radio, optical and infrared waves. Signals include man-made transient variations in amplitude, frequency, phase, polarization or other physical properties transmitted through the transmission media. Common forms of computer-readable media include, for example, a floppy disk, a flexible disk, hard disk, magnetic tape, any other magnetic medium, a CD-ROM, CDRW, DVD, any other optical medium, punch cards, paper tape, optical mark sheets, any other physical medium with patterns of holes or other optically recognizable indicia, a RAM, a PROM, an EPROM, a FLASH-EPROM, an EEPROM, a flash memory, any other memory chip or cartridge, a carrier wave, or any other medium from which a computer can read. The term computer-readable storage medium is used herein to refer to any computer-readable medium except transmission media.

While a number of embodiments and implementations have been described, the disclosure is not so limited but covers various obvious modifications and equivalent arrangements, which fall within the purview of the appended claims. Although features of various embodiments are expressed in certain combinations among the claims, it is contemplated that these features can be arranged in any combination and order.

What is claimed is:

1. A method comprising:
causing, at least in part, biometric information to be collected by way of a body-mounted device, the biometric information corresponding to a user of the body-mounted device;
causing, at least in part, the biometric information to be communicated to a network management system;
causing, at least in part, one or more of the network management system and the body-mounted device to process the biometric information to determine a status of the user of the body-mounted device; and
causing, at least in part, the status to be displayed by the body-mounted device,
wherein the status comprises one or more vital signs and an emotional state of the user based, at least in part, on the biometric information.

2. A method of claim 1, wherein the biometric information comprises one or more of a heart rate, a body temperature, a breath rate, a sweat production amount, a blood pressure, a movement, or a hydration level.

3. A method of claim 1, wherein the emotional state comprises one or more of sleeping, resting, crying, sick, happy, sad, stressed, and restless.

4. A method of claim 1, wherein the body-mounted device comprises one or more sensors configured to contact a skin surface of the user of the body-mounted device, and the one or more sensors is a dry sensor configured to collect the biometric information by way of the contact with the skin surface.

5. A method of claim 1, further comprising:
causing, at least in part, the biometric information and the status to be stored in at least one memory associated with one or more of the body-mounted device and the network management system.

6. A method of claim 1, wherein the status further comprises a determined level of pain experienced by a user of the body-mounted device.

7. A method of claim 1, further comprising:
determining food consumption of the user of the body-mounted device;
causing, at least in part, calorie intake information to be determined based, at least in part, on the determined food consumption; and
causing, at least in part, a craving log to be generated indicating a determined craving that is based, at least in part, on the determined food consumption and calorie intake information,
wherein the status further comprises the determined craving.

8. A method of claim 1, further comprising:
causing, at least in part, at least one notification to be sent to the body-mounted device based, at least in part, on the status and a rule associated with the status.

9. A method of claim 1, further comprising:
processing the determined biometric information and the determined emotional state to determine at least one suggestion for presentation to the user, the at least one suggestion being a message directing the user to follow an instruction to improve at least one of the determined biometric information or the determined emotional state; and
causing, at least in part, the message to be displayed by way of the body-mounted device.

10. A method of claim 1, further comprising:
causing, at least in part, a trend in change of status to be determined and one or more predictions based, at least in part, on the trend to be generated; and
causing, at least in part, one or more notifications based, at least in part, on the one or more predictions, to be displayed by the body-mounted device.

11. An apparatus comprising:
at least one processor; and
at least one memory including computer program code for one or more programs,
the at least one memory and the computer program code configured to, with the at least one processor, cause the apparatus to perform at least the following:
cause, at least in part, biometric information to be collected by way of a body-mounted device, the biometric information corresponding to a user of the body-mounted device;
cause, at least in part, the biometric information to be communicated to a network management system;
cause, at least in part, one or more of the network management system and the body-mounted device to process the biometric information to determine a status of the user of the body-mounted device; and
cause, at least in part, the status to be displayed by the body-mounted device,
wherein the status comprises one or more vital signs and an emotional state of the user based, at least in part, on the biometric information.

12. An apparatus of claim 11, wherein the biometric information comprises one or more of a heart rate, a body temperature, a breath rate, a sweat production amount, a blood pressure, a movement, or a hydration level.

13. An apparatus of claim 11, wherein the emotional state comprises one or more of sleeping, resting, crying, sick, happy, sad, stressed, and restless.

14. An apparatus of claim 11, wherein the body-mounted device comprises one or more sensors configured to contact a skin surface of the user of the body-mounted device, and the one or more sensors is a dry sensor configured to collect the biometric information by way of the contact with the skin surface.

15. An apparatus of claim 11, wherein the apparatus is further caused to:
cause, at least in part, the biometric information and the status to be stored in at least one memory associated with one or more of the body-mounted device and the network management system.

16. An apparatus of claim 11, wherein the status further comprises a determined level of pain experienced by a user of the body-mounted device.

17. An apparatus of claim 11, wherein the apparatus is further caused to:
determine food consumption of the user of the body-mounted device;
cause, at least in part, calorie intake information to be determined based, at least in part, on the determined food consumption; and
cause, at least in part, a craving log to be generated indicating a determined craving that is based, at least in part, on the determined food consumption and calorie intake information,
wherein the status further comprises the determined craving.

18. An apparatus of claim 11, wherein the apparatus is further caused to:
cause, at least in part, at least one notification to be sent to the body-mounted device based, at least in part, on the status and a rule associated with the status.

19. An apparatus of claim 11, wherein the apparatus is further caused to:
process the determined biometric information and the determined emotional state to determine at least one suggestion for presentation to the user, the at least one suggestion being a message directing the user to follow an instruction to improve at least one of the determined biometric information or the determined emotional state; and
cause, at least in part, the message to be displayed by way of the body-mounted device.

20. An apparatus of claim 11, wherein the apparatus is further caused to:
cause, at least in part, a trend in change of status to be determined and one or more predictions based, at least in part, on the trend to be generated; and
cause, at least in part, one or more notifications based, at least in part, on the one or more predictions, to be displayed by the body-mounted device.
